(19) United States
(12) Patent Application Publication
(10) Pub. No.: US 2010/0083391 A1
(43) Pub. Date: Apr. 1, 2010

(54) Cancer Compositions, Animal Models, and Methods of Use Thereof

(76) Inventors: Thomas C. Hamilton, Perkasie, PA (US); Stephen J. Williams, Philadelphia, PA (US); Dusica Cvetkovic, Jen Kintown, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/568,226

(22) Filed: Sep. 28, 2009

Related U.S. Application Data
(60) Provisional application No. 61/100,320, filed on Sep. 26, 2008.

(51) Int. Cl. A01K 67/00

(52) U.S. Cl. 800/10

(57) ABSTRACT

The construction of animal models of cancer and methods of using the models to screen potential cancer therapeutics are described.

This application claims priority to U.S. Provisional Application No. 61/100,320 filed Sep. 26, 2008, the entire contents being incorporated by reference herein.

### FIELD OF THE INVENTION

The present invention relates to the field of animal models for cancer. More specifically, the invention provides transformed ovarian cancer allograft and xenograft animal models, compositions, and methods of use thereof.

### BACKGROUND OF THE INVENTION

Several references and patent documents are cited throughout this application to better define the state of the art to which the invention pertains. Each of these citations is incorporated by reference herein as though set forth in full.

Animal models are important tools to investigate cancer pathogenesis and develop treatment strategies in humans. Yet a major pitfall in development of adenoviral-based chemotherapies is the lack of appropriate in vivo models for the assessment of efficacy and safety of such novel therapies. Since conditionally replicative adenoviral therapy has shown significant efficacy against several tumor types, it is imperative that suitable animal tumor models, useful for preclinical efficacy and toxicity evaluation, be developed. The instant invention provides such a model.

### SUMMARY OF THE INVENTION

In accordance with the present invention, a method for producing an animal model of cancer comprising isolating a population of cells from a donor animal, transforming said cells with a nucleic acid molecule, culturing said transformed cells, and injecting the transformed cells into a recipient animal is provided. In a particular embodiment, the recipient animal is the same species as the donor animal. In another embodiment, the recipient animal is a different species than the donor animal. In yet another embodiment the recipient animal is genetically identical to the donor animal. Preferably, the donor and recipient animals are pigs (e.g., miniature swine) and the cells are porcine ovarian surface epithelial cells resulting in an animal model of ovarian cancer. In still another embodiment, the transformation of cells is performed with the Sleeping Beauty transposon system and the nucleic acid molecule encodes the SV40 T-antigen.

In another aspect of the invention, an animal model produced according to the methods described herein is provided. In yet another aspect, an animal comprising an allograft or xenograft of carcinoma is provided. A preferred embodiment comprises ovarian carcinoma comprising ovarian epithelial cells transformed with SV40 T antigen. In another aspect, the animal is of porcine origin. In still an additional aspect, the ovarian carcinoma is a xenograft or allograft.

The methods of the invention also include identifying active therapeutic agents for cancer comprising providing an animal model of cancer, administering at least one agent to said animal, and determining the effect of the agent(s) on the tumor of the animal, wherein a decrease in the size of the tumor compared to a control animal not administered the agent(s) indicates the administered agent is an active therapeutic agent. In another embodiment, the agent comprises an adenovirus, preferably an oncolytic conditionally-replicative adenovirus or CRAd. In yet another embodiment, a method for identifying a chemotherapeutic agent comprising providing an animal model of cancer, and administering at least one agent to said animal, wherein an increase in the survival time of the animal compared to a control animal not administered the agent(s) indicates the administered agent(s) is an effective chemotherapeutic agent, which can comprise an adenovirus.

### BRIEF DESCRIPTION OF THE DRAWINGS

FIG. 1. Scheme for sub-culturing Sleeping Beauty-T Antigen (SB-TAg) transfected porcine ovarian surface epithelial (POSE) cells.

FIG. 2. Transformation of POSE cells with T-antigen using the Sleeping Beauty system. (A) Freshly isolated POSE cells showing cobblestone epithelial morphology, (B) Passage 3 after SB-TAg transfection, (C) Passage 4, (D) Foci formation at passage 6. (40× magnification), (E) High magnification of confluent POVCAR cells at passage 6.

FIG. 3. (A) Validation of TAg expression in POSE cells by Western blot. 20 μg cell lysate was subjected to SDS-PAGE and probed for TAg and GAPDH protein. Blot repeated twice with similar results. (B) Verification of species of origin of POVCAR cells by genomic PCR. A porcine specific genomic marker was used to amplify a 212 base pair product from 100 ng of genomic DNA. DNA from Lane 1: POVCAR cells (passage 8), 2: Normal untransformed POSE cells, 3: Normal mouse ovarian surface epithelial (MOSE) cells, 4: Human ovarian surface epithelial (HOSE) cells, 5: water input control. PCR for genomic GAPDH was used as loading control.

FIG. 4. POVCAR clone in soft agarose. To demonstrate anchorage-independent growth, 2×10⁵ cells were embedded in soft agarose and allowed to grow for 14 days as described in Materials and Methods. (A) Normal, untransformed POSE cells, show no ability to clone in soft agarose. (B) POVCAR cells display high colony forming efficiency (−0.2%). (C) 40× magnification of individual colonies in FIG. 4B.

FIG. 5. In vivo growth of POVCAR cells in SCID mice. Five million cells were injected s.c. into flanks of SCID mice. Arrow shows tumor formation one month after s.c. inoculation with cells. Tumor volume was measured using caliper and volume calculated via the formula (volume=½×length×(width²)). Volume of tumor depicted was 270 mm³ (8.75 mm×7.87 mm). The set of six panels represent H&E staining and IHC analysis for TAg and cytokeratin, respectively. Lower panels show a 40× magnification of area indicated by yellow box in upper panel (10× magnification).

FIG. 6. POVCAR cells are highly permissive to adenovirus serotype 5 replication and genetically modified vectors can be targeted to POVCAR cells and have reduced targeting to porcine liver. (A) POVCAR cells are highly permissive to adenovirus serotype 5 replication. To measure adenoviral serotype 5 replication, 5×10⁴ cells/well were plated into a 24-well cell culture plate and incubated overnight. The next day, the cells were infected with 100 vp/cell of native adenovirus serotype five. At days 0, 1, 3, 6 and 9, copies of adenoviral E4 region of the genome were measured in the cell culture media by duplexing real-time PCR. Known amounts of target DNA (10⁸, 10⁶, 10⁴ and 10² copies/μl) were amplified to generate a standard curve for quantification of the target gene copy numbers. (B) Adenovirus containing the knob domain of porcine adenovirus preferentially infects POVCAR cells compared to porcine liver cells. Adenoviral vectors with various capsid modifications expressing the reporter gene luciferase were incubated with POVCAR cells or porcine liver cells at 100 vp/cell. Forty-eight hours post-infection, cells were harvested and lysed using cell culture lysis buffer and luciferase was measured. (C) The CXCR4 and survivin promoter regions allow for transgene expression in POVCAR cells and limited expression in porcine liver. Adenoviral vectors with various promoter elements controlling the expression of the reporter gene luciferase were incubated with POVCAR cells or porcine liver cells at 100 vp/cell. Forty-eight hours post-infection, cells were harvested and lysed utilizing cell culture lysis buffer and luciferase was measured. Virus nomenclature: Ad5-Luc.RGD—adenovirus serotype 5 (Ad5) containing luciferase reporter gene (Luc) and integrin binding peptide RGD. Ad5-Luc.F5/3—contains Ad5 fiber and Ad3 knob domain. Ad5-Luc.sigma-1—knob contains sigma-1 domain from reovirus. Ad5/3.sigmal—contains both Ad5 fiber, Ad3 knob, and sigma domain from reovirus on knob. Ad5-Luc.CK-1—contains canine adenovirus serotype one knob domain. Ad5-Luc.CK2pk7—contains canine adenovirus serotype two knob and pk7 motif. Ad5-Luc.OVF—contains ovine fiber domain. Ad-Luc.PF—contains porcine adenovirus fiber. Ad5-Luc.PK—contains porcine knob domain. Ad5-Luc.D.RGD—contains two RGD motifs (knob and pIX capsid protein). Ad5-Luc.pk7—knob domain contains pk7 motif. Ad5-Luc.RGDpK7—contains RGD and pK7. Ad5-Luc.CXCR4—Ad5 with luciferase under control of CXCR4 promoter. Ad5-Luc.Survivin—Survivin promoter. Ad5-Luc.COX2—cycloxygenase two promoter. Ad5-Luc.EGP-2—Epithelial cell adhesion molecule two promoter. Ad5-Luc.HPR—heparinase promoter. Ad5-Luc.Msln—mesothelin promoter. Ad5-Luc.SLP1—Stomatin promoter. Ad5-Luc.MK—midkine promoter. Ad5-Luc.Robo—Roundabout factor four promoter.

### DETAILED DESCRIPTION OF THE INVENTION

Results in porcine models resemble human results in terms of adenoviral replication. Hence, the present inventors have developed an ovarian porcine tumor model useful in studies of ovarian-specific adenoviral therapies. This concept is supported by data that human adenoviruses can replicate in pigs and the similarities between porcine adenovirus and human adenovirus.

In a particular embodiment of the instant invention, miniature swine, made tolerant to T-antigen (T-Ag or TAg), can be inoculated with the porcine tumor cells intraperitoneally. The titer of adenovirus (e.g., multiplicity of infection (MOI)) to be used for in vivo testing may be determined in vitro using the isolated porcine tumor cells. Briefly, porcine tumor cells would be infected with increasing MOI of human adenovirus, and inhibition of growth determined by short-term proliferation assays. Subsequently the swine would be inoculated with optimal MOI of human adenovirus and monitored for signs of physical distress and ascites production. Serum hepatic enzyme levels can be monitored for the determination of hepatotoxicity, as well as other methods known to the skilled artisan to assess the treatment.

The results shown hereinbelow demonstrate the generation of a xenograft model of the instant invention in mice. However, the instant invention encompasses xenograft and allograft models of any animal. In a preferred embodiment of the invention, the allograft or xenograft model is porcine. Particularly, the instant invention encompasses an allograft of porcine ovarian carcinoma cells (POVCAR) cells inoculated (i.e., injected) into a pig. This would allow for the testing of efficacy and toxicity of agents, particularly adenoviral and adeno-associated viral delivery methods, for cancer therapeutics in ovarian cells.

Additionally, in light of the demonstration herein concerning the ability to transform porcine ovarian epithelial cells, the invention contemplates transformation of other porcine cell types, such as mammary, prostate, lung, colon, skin, uterus, and esophagus using the Sleeping Beauty T-Ag transposon system described herein.

In general, tumor xenograft experiments must be done in an immunocompromised animal to limit immune rejection of the tumor. However, in the BL6 mouse expressing low T-Ag levels, it is possible to propagate syngeneic mouse T-Ag expressing tumor cells in the intraperitoneal cavity. With regard to the instant invention, ovarian surface epithelial cells from an inbred porcine strain can be transformed using the T-Ag system. These transformed cells can then be propagated in a low-T-Ag expressing porcine strain or pigs made tolerant of T-Ag, as with the above BL6 mouse model. Recent advances in the development of genetically-modified, inbred-porcine strains also aid in the ability to perform syngeneic transfer of transformed cells into pigs. To this effect, a highly inbred miniature swine has been developed that achieve stable engraftment across major histocompatibility complex (MHC) haplotypes without graft-versus-host-disease. Researchers have shown that porcine tumor cells can be transplanted into these miniature swine (Cho et al. (2007) Blood, 110:3996-4004). Accordingly, the instant invention encompasses an allograft or xenograft model of these miniature swine.

Furthermore, it is desirable to utilize an inbred pig strain in practice of the invention, for example, the Westran pig described in O'Connell et al. (Xenotransplantation (2005) 12:308-315). In particular, it is preferable that the pig has low expression of T-Ag, but will not develop tumors. Sufficiently inbred pig strains could receive an allograft of transformed cells from another pig of that strain after the recipient was made tolerant to T-Ag by neonatal exposure.

In the absence of an appropriate inbred pig strain, an embryo splitting technique could be employed to yield genetically identical pigs. Briefly, pig embryos would be split and divided in the first step. Subsequently, some of the embryos would be implanted into female pigs, and the pigs that the implanted embryos give rise to would be sacrificed and the ovaries harvested. Ovarian epithelial cells would then be isolated from the harvested embryos, cultured, and transformed as described herein. At this stage, the tumorigenicity of the isolated cells may be assessed in SCID mice. Remaining embryos from the initial split, which are genetically identical to the isolated transformed cells, would be implanted into female pigs. These neonatal pigs would be tolerized to T-Ag, and would be implanted with the genetically-related ovarian epithelial cells that were transformed resulting in an allograft model of cancer that could be utilized in drug discovery and other experiments described herein. One example of embryo splitting is described in Chan et al. (Science (2002) 287: 317-319).

In yet another approach, genetically identical animals can be produced using somatic cell nuclear transfer (SCNT), also referred to as cloning. This technique was used to generate Dolly the sheep in 1997 and has since been applied to pigs, cows, and numerous other species. The SCNT procedure is now routine for many laboratories. For nuclear transfer, an unfertilized egg is treated with cytoskeletal inhibitors to depolymerize the microtubules and/or microfilaments and impart elasticity to the plasma membrane. A beveled micropipette is then inserted through the zona pellucida. Since the plasma membrane is very elastic, it invaginates around the pipette. The chromosomes are located, aspirated into the pipette, and when the pipette is withdrawn, the zona pellucida pinches off the cell membrane. This produces a membrane-bound nucleoplast in the pipette and an oocyte with no nucleus/chromosomes. A nuclear donor cell is then placed within the zona pellucida next to the recipient ovum. Viral, chemical, or electrical stimuli are then used to induce the fusion process between the two cells. Alternatively, a donor nucleus can be microinjected directly into the cytoplasm of the recipient oocyte. These procedures deposit the nucleus into the cytoplasm of the recipient ovum and mix the cytoplasmic contents of the two cells. There can be many technical variations on the above description (reviewed in Prather et al. (1999) Theriogeneology 51:487-498).

After nuclear transfer, subsequent development requires activation of the ovum. The mammalian ovum is generally arrested at metaphase II of meiosis. Fertilization breaks this arrest and activates oocyte development. When performing nuclear transfer it is necessary to artificially break this meiotic arrest, otherwise the nuclear transfer recipient embryo will remain arrested at the one-cell stage with condensed chromosomes. Artificial activation of meiosis can be accomplished with the electrical pulse used for fusion of the donor cell with the recipient oocyte, or chemically. See Prather et al. (supra).

After the nucleus is transferred into the cytoplasm of the recipient ovum, the nuclear envelope breaks down and the chromosomes condense. Proteins within the nucleus migrate into the cytoplasm, and cytoplasmic proteins assemble in the newly formed nucleus. This nuclear-cytoplasmic exchange of proteins reconfigures nuclear structure such that RNA synthesis is altered. If the nucleus is truly reprogrammed, it will reinitiate its developmental pathway and recapitulate early developmental events of the embryo (Prather et al. (2003) Theriogenology 59:115-23; Prather et al. (2000) Science 289:1886-1887). As a result, some of the embryos will develop to term when transferred to a surrogate.

We have chosen to develop this model in a miniature pig line, more specifically the Yucatan miniature pig. While it possesses the same biological characteristics as domestic pigs, the Yucatan mini pig is significantly smaller. Most domestic pig breeds reach 100 kg in less than six months and can achieve weights of 250-300 kg within a few years. Yucatan miniature pigs reach a full-grown size of 65-90 kg, more similar to an adult human. Therefore, the Yucatan miniature pigs are less expensive to house and feed. Additionally, they are more docile in nature and better suited for interactions with researchers (Panepinto et al. (1986) Lab Animal Sci. 36:344-347).

To date, the testing of new adenoviral therapies has been performed solely in human cancer cell lines and human tumor xenografts in immunodeficient mice. However, the use of human tumor xenografts in immunodeficient mice presents a problem for ovarian cancer studies given the lack of proper host-tumor microenvironment, thereby limiting pharmacokinetic and toxicity studies. One major concern with adenoviral therapy in humans is the development hepatic toxicity. However, hepatic toxicity is not seen in rodent models since human adenovirus does not infect and propagate in rodent tissues. Recently, an ex vivo system using human hepatic tissue slices has been used to determine hepatic toxicity, but this strategy is limited by the availability of tissues and does not allow pharmacokinetic issues to be addressed.

Another important therapeutic approach that must be strenuously evaluated for the treatment of ovarian cancer includes the use of oncolytic conditionally-replicative adenovirus (CRAd). CRAd oncolytic virotherapy utilizes the cancer cell's own transcription machinery to create progeny virions, lyse the cancer cells, and spread to neighboring cancer cells. This highly attractive approach (Chiocca et al. (2002) Nat Rev Cancer 2:938-950) was initially pursued to address the problems experienced with earlier cancer gene therapy strategies such as development of resistance and high levels of toxicity and side effects (Alemany et al. (2000) Nat. Biotechnol. 18:723-7; Kirn et al. (2001) Nat. Med. 7:781-7.

Although earlier attempts met with liver toxicity and efficacy issues, the development of genetically engineered, conditionally-replicating adenoviruses (CRAds) have increased the tumor specificity and host safety dramatically. The improved features of these “targeted” CRAds include enhanced tumor transduction and specificity, which prevents the virus from entering and replicating in normal tissues.

To accomplish efficient transduction, CRAds depend on initial binding to the coxsackie-adenoviral receptor (CAR), the natural receptor for adenovirus serotypes 2 and 5 (Ad2 and Ad5) (Li et al. (1999) Cancer Res. 59:325-30; Miller et al. (1998) Cancer Res. 58:5738-48) which is usually expressed at low levels on most tumors. Engineered modifications to the viral capsid, which mediates cell binding and entry, have improved infectivity of Ad5-based CRAds. In doing this, adenovirus-based CRAds can be targeted to alternate receptors and have been employed successfully. See Curiel et al., (1999) Ann N Y Acad. Sci. 886:158-71; Douglas et al. (1996). Nat. Biotechnol. 14:1574-8; and Krasnykh et al. (1996) J. Virol. 70:6839-46.

Complex mosaic viruses have been generated which have improved infectivity by virtue of additive effects of dual-receptor targeting within a single fiber in the viral capsid. The ability of the virus to bind multiple cellular receptors significantly increases entry of adenovirus into cancer cells, but infection of some normal tissues such as liver is also increased. To overcome this limitation, new CRAds utilize tumor-specific promoters (TSP) for controlling the crucial Ad5 genes required for virus replication, such as E1A. Combining these strategies increases infectivity of tumors with viral replication and cell lysis limited to tumor cells by the TSP. Specifically, the CXCR4 gene promoter element, which is highly expressed in many cancer types, demonstrates improved selectivity and high replication efficiency in breast and ovarian cancer. Similar CRAd targeting strategies using tumor/tissue specific promoters have advanced quickly into human clinical trials and are demonstrating clinical efficacy.

Although new-generation CRAds present with a relatively safe profile, adenoviral particles, especially the Ad5-based virus used for most CRAds, may replicate in non-tumor tissue at low levels. This can result in tissue-specific toxicities, mainly in liver and lung. These toxicities can only be accurately assessed in species permissive for viral replication, and mice and many other common cancer models are not permissive for Ad5 replication. In addition, human Ad5 elicits immune responses, which limit bioavailability of the adenoviral vector. Thus, testing in immunodeficient models does not yield relevant data on the reaction of the immune system to the vector and the impact of immunity on the efficacy of treatment. A major limitation of preclinical evaluation of CRAds has been the lack of immunocompetent animal cancer models permissive for adenoviral replication. Indeed, one of the first human trials with untargeted adenoviral vectors, resulted in severe hepatic toxicity and fatality, and this event may have been prevented if more appropriate preclinical screens were conducted in an immunocompetent model permissive for viral replication in the liver and other tissues. Since then, strict regulatory guidelines for adenoviral-based clinical trials have been issued, with particular emphasis on vector dosage, safety, and toxicity. But the field has remained hampered by the lack of an immunocompetent animal model that is permissive for adenoviral replication.

In this regard, only a few species have been identified that are permissive to human Ad5 virus. Mammalian cells permissive for Ad5 include the Syrian hamster, cotton rat, dog, hen, and pig. The cotton rat and Syrian hamster have been used to test adenovirus in syngeneic, immunocompetent cancer models, as well as adenoviral-related toxicity. However a screen of these and other species revealed that the permissiveness of porcine cells most resembled that of human. Indeed, at Schering-Plough a toxicology program was initiated to evaluate SCH 58500, an adenoviral gene therapy directed against p53, which involved use of non-immunogenic rats compared with Yorkshire pigs made immunoreactive to the vector. Data from the study revealed faster clearance of virus and toxicities in the pig not seen in non-immunogenic, non-permissive hosts such as rat and mouse.

Therefore, development of an allograft ovarian tumor model in a fully-permissive host would be ideal for evaluation of CRAd efficacy, toxicity, and biodistribution. Due to the permissive nature of porcine cells to Ad5, similarities to human anatomy and physiology, and opportunities for genetic manipulation, the miniature pig seems ideal as a preclinical model for testing of these compounds. Development of novel adenoviral based CRAd therapies in this strict animal model may provide the key relevant data to overcome obstacles to advances in CRAd based therapy.

Thus, the instant animal model system described herein alleviates many issues related to preclinical testing of adenoviral therapies, most importantly, the ability to test toxicity profiles in an animal model closely related to human. The transformed porcine cells would be able to grow in a syngeneic host. Therefore, both pharmacodynamic, pharmacokinetic, and toxicity studies could be carried out in vivo in one large animal system. In addition, use of a syngeneic model permits chronic studies to be performed, which would be a requisite for regulatory approval. Human adenoviruses are known to propagate in porcine tissues such as liver, so potential hepatic toxicity of future therapies can be directly evaluated.

In developing the present invention, several factors were found to be important to the new and unexpected properties of the resulting cancer model. One such factor is compatibility; the porcine genome is remarkably similar to the human as compared to other species, such as mouse. In addition, as alluded to above, the ability of human adenovirus to infect porcine tissue confers the ability to test human oncoviral therapies in a porcine-derived tumor model. Therefore, results from chronic and subchronic studies would be more useful for human risk-assessment before the onset of human clinical trials. As proof of principle, a transformed, porcine ovarian epithelial cell line was developed ex vivo for studies in animals (e.g., immunosuppressed pigs).

Another factor found to be important for implementing the present invention on a large scale is availability of the animal (e.g., pig) to receive the tumor graft. Pigs are easily bred and, with the advances in pig genetic engineering, the syngeneic porcine tumor models can be readily produced on a large scale with nominal cost. In addition, the production of a transformed cell line from inbred pigs or genetically identical pigs, based on the demonstrated success with outbred pig ovaries, will allow for easy culturing, the ability to freeze and stock, and will allow for easy transfer to other interested parties.

Reliability is another important feature of the present invention. Previous studies with spontaneous derived porcine tumor cells have not shown reliable tumorigenic potential over serial passages and multiple freeze/thaws. This provides one clear advantage of developing an ex vivo transformed cell line. In addition, the system described herein allows for stable, continuous expression of the T-Ag over multiple passages, thereby creating a more stable tumor cell line with extended resourcefulness in animal models.

### I. DEFINITIONS

The following definitions are provided to facilitate an understanding of the present invention:

A “clone” or “clonal cell population” is a population of cells derived from a single cell or common ancestor by mitosis. A “cell line” is a clone of a primary cell or cell population that is capable of stable growth in vitro for many generations.

By “construct” is meant a recombinant nucleic acid, generally recombinant DNA, which has been generated for the purpose of the expression of a specific nucleotide sequence(s), or is to be used in the construction of other recombinant nucleotide sequences. Exemplary constructs are pCMV-SB containing the transposase, and SB-TAg containing the SV40 T antigen.

The term “animal” is used herein to include all vertebrate animals, except humans. It also includes an individual animal in all stages of development, including embryonic and fetal stages. A “animal” or “animal model” is any animal (e.g., mouse, rat, rabbit, pig, etc.) containing one or more cells bearing genetic information altered or received, directly or indirectly, by deliberate genetic manipulation at the subcellular level, such as by targeted recombination or microinjection or infection with recombinant virus. The animals can be used to assay or test agents (e.g., an adenoviral construct, a candidate drug or compound) for efficacy on cancer development and progression, or in samples or specimens (e.g., a biopsy) from the animals. In some instances, it is advantageous to measure markers of cancer progression in samples, for example, blood, which can be obtained from the test animal without the need to sacrifice the animal. The term “animal” or “animal model” is not meant to encompass classical cross-breeding or in vitro fertilization, but rather is meant to encompass “host” animals in which one or more cells are altered by or receive a recombinant DNA molecule or a graft containing the same. This molecule may be specifically targeted to defined genetic locus, be randomly integrated within a chromosome, or it may be extrachromosomally replicating DNA. In particular, the gene of interest can be introduced to the cell using the Sleeping Beauty transposon-transposase system described herein.

As used herein “cancer model” or “model of cancer” refers to an animal exhibiting uncontrolled cellular growth in one or more tissues or organs leading to the formation of precancerous lesions and/or cancer that result in tumor formation. A non-human animal cancer model can exhibit, for example, hyperplasia, dysplasia, in situ carcinoma, invasive neoplasias and/or metastatic cancer.

In a particular aspect of this invention, the recipient animal is a non-human mammal capable of receiving and supporting a graft. Particularly, the recipient animal is capable of receiving an allograft. Alternatively, the animal can receive a xenograft by being immuno-compromised and is mostly incapable of mounting a graft-rejection immune response thereby accepting the foreign tissue as self. The recipient animal can be immuno-compromised either by being immuno-deficient or immuno-suppressed by biological or chemical means. Such biological or chemical means include, without limitation, immuno-suppression by repeated treatment with cyclosporin or other immuno-suppressive agents well known in the art. More preferably, the immuno-compromised animal is immuno-deficient. The term immuno-deficient is used to describe a recipient animal in which the immune system has been partly or completely compromised in order to allow engrafted foreign cells or tissue to grow with minimal chance of rejection by the recipient animal.

The terms “transformation”, “transfection”, and “transduction” refer to methods of inserting a nucleic acid and/or expression construct into a cell or host organism. These methods involve a variety of techniques, such as treating the cells with virus (e.g., retrovirus), high concentrations of salt, an electric field, cationic lipids, microinjection, PEG-fusion, or detergent, to render the host cell outer membrane or wall permeable to nucleic acid molecules of interest. “Transformation” encompasses a permanent or transient genetic change, in some embodiments a permanent genetic change, induced in a cell following incorporation of new DNA (i.e., DNA exogenous to the cell), for example, T-Ag. Where the cell is a mammalian cell, a permanent genetic change is generally achieved by introduction of the DNA into the cell. In particular, T-Ag is known to affect the activity of the tumor suppressor genes p53 and Rb.

As used herein, the term “agent” denotes a chemical compound, a mixture of chemical compounds, a biological macromolecule, or an extract made from biological materials such as bacteria, plants, fungi, or animal (particularly mammalian) cells or tissues. Agents also encompass proteins, polypeptides, nucleic acids, and vectors such as adenoviruses and adeno-associated viruses and vectors containing the same. Agents can be evaluated for potential biological activity by inclusion in screening assays described hereinbelow. Potential therapeutic agents may be tested alone or in combination to identify pharmaceutical compositions that act synergistically to kill tumor cells. Such agents, include, without limitation, (according to the general classes of the compounds): Alkylating agents: Nitrogen mustards (mechlorethamine; cyclophosphamide; ifosfamide; melphalan; chlorambucil); Nitrosoureas (carmustine (BCNU); lomustine (CCNU); semustine (methyl-CCNU)); Ethylenimine/Methylmelamine (thriethylenemelamine (TEM); triethylene thiophosphoramide (thiotepa); hexamethylmelamine (HMM, altretamine)); Alkyl sulfonates (busulfan); Triazines (dacarbazine (DTIC)); and Antimetabolites (Folic Acid analogs—methotrexate and trimetrexate; Pyrimidine analogs-1-5-fluorouracil, fluorodeoxyuridine, gemcitabine, cytosine arabinoside, 5-azacytidine, 2,2′-difluorodeoxycytidine); Purine analogs—6-mercaptopurine, 6-thioguanine, azathioprine, 2′-deoxycoformycin (pentostatin), erythrohydroxynonyladenine (EHNA), fludarabine phosphate, and 2-Chlorodeoxyadenosine (cladribine, 2-CdA)); Type I Topoisomerase Inhibitors: camptothecin; topotecan; irinotecan; Natural products: Antimitotic drugs (paclitaxel; Vinca alkaloids—vinblastine (VLB), vincristine, and vinorelbine; Taxoter® (docetaxel); estramustine; estramustine phosphate); Epipodophylotoxins (etoposide and teniposide); Antibiotics (actimomycin D; daunomycin (rubidomycin); doxorubicin (adriamycin); mitoxantrone; idarubicin; bleomycins; plicamycin (mithramycin); mitomycinC; and dactinomycin); Enzymes (L-asparaginase); Biological response modifiers: interferon-alpha; IL-2; G-CSF; and GM-CSF; Differentiation Agents: retinoic acid derivatives; Radiosensitizers: metronidazole, misonidazole, desmethylmisonidazole, pimonidazole, etanidazole, nimorazole, RSU 1069, E09, RB 6145, SR4233, nicotinamide, 5-bromodeozyuridine, 5-iododeoxyuridine, bromodeoxycytidine, Miscellaneous agents: Platinum coordination complexes (cisplatin, carboplatin); Anthracenedione (mitoxantrone); Substituted urea (hydroxyurea); Methylhydrazine derivatives (N-methylhydrazine (MIH) and procarbazine); Adrenocortical suppressant (mitotane (o,p′-DDD) and aminoglutethimide); Cytokines (interferon (α, β, γ) and interleukin-2); Hormones and antagonists: Adrenocorticosteroids/antagonists (prednisone and equivalents; dexamethasone; aminoglutethimide); Progestins (hydroxyprogesterone caproate; medroxyprogesterone acetate; megestrol acetate); Estrogens (diethylstilbestrol, ethynyl estradiol/equivalents); Antiestrogen (tamoxifen); Androgens (testosterone propionate, fluoxymesterone/equivalents); Antiandrogens (flutamide; gonadotropin-releasing hormone analogs, leuprolide); Nonsteroidal antiandrogens (flutamide); Photosensitizers: hematoporphyrin derivatives, Photofrin®, benzoporphyrin derivatives, Npe6, tin etioporphyrin (SnET2), pheoboride-a, bacteriochlorophyll-a, naphthalocyanines, phthalocyanines, and zinc phthalocyanines; Proteosome inhibitors: bortezomib (Velcade®). In addition to the above, there are several novel compounds disclosed in various patent applications that are contemplated as second therapeutic agents, e.g. combretastatins from Bristol Myers Squibb, Epothilones (US 2005244413), serratamolide (US 2005239694), indol derivatives (US 2005239752), various plant extracts: extract of sea buckthorn—Hippophae rhamnoides (US 2005214394), extracts of Ganoderma lucidum, Salvia miltiorrhiza and Scutellaria barbata (US 2005208070), chk1 inhibitors (WO 2006/021002; WO/2006/014359; WO 2006/012308; WO 2005/027907; WO 2002/070494; WO 1999/011795); the contents of the afore-mentioned Patents and Patent Applications are herewith incorporated by reference in their entireties. Also suitable are tyrosine kinase inhibitors. Specific tyrosine kinase inhibitors include, but not limited to imatinib mesylate (marketed as Gleevec or Glivec; previously known as STI-571), dasatinib, nilotinib, MK-0457 (formerly known as VX-680), and Omacetaxine (formerly known as Ceflatonin). Antibodies which impact importance cellular proliferation pathways may also be tested, including herceptin, and those targeting the EGFR receptor such as cetuximab etc.

Generally, the effect of at least one compound (agent) on a test animal is compared with a test animal in the absence of the test compound. Test compounds considered to be positive are likely to be beneficial in the treatment of cancer. The test compounds or agent can be any molecule, compound, or other substance which can be administered to a test animal. Suitable test compounds may also be small molecules, biological polynucleotides, cells and the like. Preferably, the animals of the instant invention can be used to test safety and efficacy of nucleic acids delivered to the animal. The nucleic acids can be delivered by methods known to the skilled artisan, preferably via adenovirus or adeno-associated virus to the site of the allograft or xenograft where a tumor is located.

A “therapeutic agent” as used herein refers to an agent that can mitigate, cure, treat or prevent a disease or condition. It is particularly desirable that the therapeutic agent be capable of exerting it effect locally (i.e., at or near the site of the disease or condition). Exemplary therapeutic agents include, but are not limited to, antibiotics, anti-proliferative agents, anti-neoplastic agents, chemotherapeutic agents, cardiovascular agents, anti-inflammatory agents, immunosuppressive agents, anti-apoptotic and anti-tissue damage agents.

The term “treating”, “treat” or “to treat” as used herein means the prevention, reduction, partial or complete alleviation or cure of a disease. Preferably the disease is cancer.

The term “delivery” as used herein refers to the introduction of foreign molecule (i.e., protein containing nanoparticle) into cells. The term “administration” as used herein means the introduction of a foreign molecule into a cell. The term is intended to be synonymous with the term “delivery”. Administration also refers to screening assays of the invention (e.g., routes of administration such as, without limitation, intravenous, intra-arterial, intramuscular, subcutaneous, intrasynovial, infusion, sublingual, transdermal, oral, or topical). The preferred method of delivery is application to the tumor cells, or, in particular, intra-tumoral or intraperitoneal injection.

A “sample” or “patient sample” or “biological sample” generally refers to a sample which may be tested for a particular molecule, preferably polynucleotide, polypeptide, or antibody. Samples may include but are not limited to cells, tissue, and body fluids, including blood, serum, plasma, cerebral-spinal fluid, urine, saliva, pleural fluid and the like. A “patient” or “test subject” refers to an organism which may be tested for a particular disorder, including but not limited to a disorder characterized by uncontrolled cellular proliferation. A “test subject” includes, but is not limited to animals, including mammalian species such as murine, porcine, ovine, bovine, canine, feline, equine, human, and other primates.

### II. CELL LINES

The present invention provides ovarian cancer cell culture lines derived from animals (see, e.g., the Example hereinbelow). The cell lines of the invention may be used to identify anti-proliferative agents in methods which utilize the instant animal model systems. In particular embodiments, cell lines exposed to a test agent (e.g., a potential cancer therapeutic) may be assayed for efficacy and toxicity. For example, the cell lines of the invention may be exposed to T-Ag, evaluated for transformation, and subjected to testing various compounds and therapeutic intervention, for example, adenoviral therapies. A dose-response relationship between test agent concentration and percent of tumor reduction may be established for a given agent and then compared to the dose-response relationship of known therapeutic to reduction in tumor size.

### III. ANIMALS HARBORING TRANSFORMED CELLS

Cancer-susceptible animals of the invention include animals that carry a xenograft or allograft of transformed cells. The cells can be spontaneously transformed or transformed by an oncogene; preferably the SV40 T-Ag is used in the transformation of cells. The oncogene can be introduced via any method, such as the SB-TAg transposon system described herein.

In addition, carcinomas and other lesions arising in the animals of the invention, or corresponding cell lines, may be analyzed for changes in specific gene expression during the progression of malignancy, including increases and decreases in gene expression. Such analyses may be performed using standard techniques such as PCR, immuno-assay, and other techniques known to the skilled artisan.

In a particular embodiment of the present invention, there is provided a model for grafting tissue from a donor animal that is the same species (allograft) or of foreign species (xenograft). As stated above, when the graft is foreign (xenograft) the recipient animal should be immuno-compromised to be able to support such graft without rejecting it as non-self. This is exemplified in the Example wherein porcine cells are injected into a mouse. However, in another embodiment of the invention there is provided an allograft animal model capable of forming tumors in the recipient animal (i.e., transformed porcine cells grafted into a pig).

### IV. ASSAYS

In further embodiments, the animals of the invention may be used to identify agents that inhibit carcinoma formation and/or progression. Agents that inhibit carcinoma may be used in prophylactic treatment regimens or in the therapy of existing carcinoma. For example, p53 and RB are perturbed by the SV40-T-antigen oncogene. A compound being tested may be delivered to the animal and the expression of these putative tumor suppressor genes can be analyzed and compared to expression in control animals. The agent or compound can be administered by any means including, without limitation, parenterally, i.e., via an intraperitoneal, intravenous, perioral, subcutaneous, intramuscular, intraarterial, and the like. The compound may also be administered directly to the tumor (e.g., injection). The agent may also be contained within a pharmaceutically acceptable carrier. The agent, compound, or compositions can comprise adenoviral vectors or adeno-associated viral vectors.

The following example has been included to illustrate an exemplary mode of the presently disclosed subject matter. Certain aspects of the following example are described in terms of techniques and procedures found or provided by the present inventors to work well in the practice of the presently disclosed subject matter. This example is exemplified through the use of standard laboratory practices of the inventors. In light of the present disclosure and the general level of skill in the art, those of skill will appreciate that the following example is intended to be exemplary only and that numerous changes, modifications, and alterations can be employed without departing from the scope of the presently disclosed subject matter.

### Example

The following materials and methods are used to facilitate practice of the instant invention. The methods and techniques are generally performed according to conventional methodology known in the art and described in the references cited or, for example, Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), which are incorporated by reference herein.

### Porcine Ovarian Surface Epithelial (POSE) Cell Isolation

Ovaries from 8-12 week old Yorkshire and/or Devonshire pigs were obtained from Spear Products (Spear Products, Inc, Trevose, Pa.) after gross dissection at the slaughterhouse and immediate placement on ice. All pigs were deemed disease-free following post-mortem analysis by a certified veterinarian. Ovaries were then transferred to sterile specimen cups containing sterile, ice-cold calcium-containing phosphate-buffered saline (Ca⁺⁺-PBS), supplemented with 250 ng/ml fungizone and 10 μg/ml ciprofloxacin. In general, four ovaries were obtained for typical cell isolation. Upon arrival at the laboratory, ovaries were thoroughly washed with Ca⁺⁺-PBS (supplemented with 250 ng/ml fungizone and 10 μg/ml ciprofloxacin) and excess tissue was aseptically removed. Ovaries were then washed three times with 10 ml calcium-free PBS (PBS, supplemented with 250 ng/ml fungizone and 10 μg/ml ciprofloxacin) to remove visible blood. Three successive trypsinizations were used to remove POSE cells. In detail, each ovary was transferred to a clean specimen cup containing 20 ml of 0.25% trypsin-0.1 mM EDTA and incubated at 37° C. for 30 minutes. The trypsin solution was removed and saved and an additional 20 ml of fresh trypsin solution was added, and incubated for a further 15 minutes. At the end of the second successive incubation period, the solution was removed and saved and another 20 ml of trypsin solution was added and a further incubation for 15 minutes was performed. This final solution was also saved. All three trypsin incubations were centrifuged separately, and each plated in an individual well of a 6-well dish, 10 cm²/well (Fisher Scientific, Pittsburgh Pa.) in Dulbecco's Modified Eagle Medium containing 4% fetal bovine serum, 2 mM glutamine, 100,000 units/liter of both penicillin and streptomycin, 250 ng/ml fungizone, 10 μg/ml ciprofloxacin, 10 μg/liter insulin, 5.5 mg/liter transferrin, and 6.7 μg selenium (Mediatech Inc., Manassas Va.) at 37° C., 5% CO₂. Cells were allowed to attach for two days and then washed with sterile PBS to remove all blood cells. POSE cells grew as isolated patches (islands) of cells and were visible on day two. The second trypsinization yielded the greatest number and purest population of POSE cells; this population was used for subsequent transfections with the Sleeping Beauty-TAg system.

Transfection with SB-TAg Transposon System

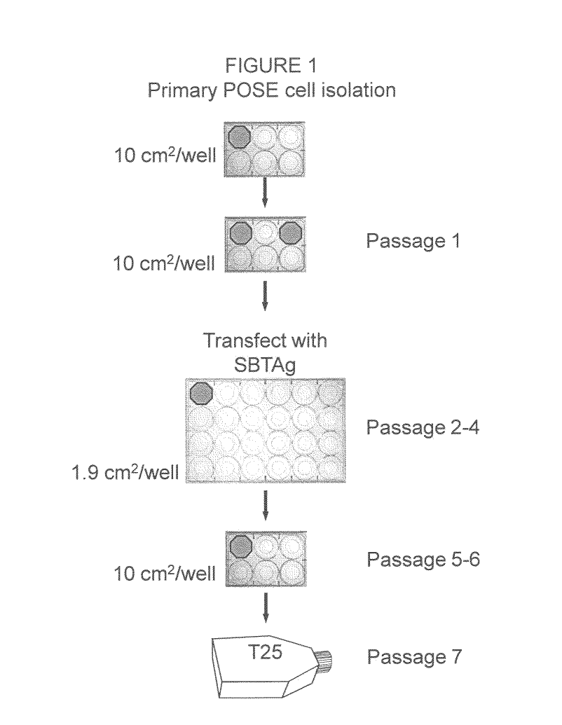

The Sleeping Beauty transposon-transposase system is described in detail elsewhere (Roberg-Perez et al. (2003) Nuc. Acids Res., 31:78-81; Izsvak et al. (2000) J. Mol. Biol., 302:93-102). Briefly, the system consists of two vectors, one encoding a transposase, and the other containing the transposable element where the gene of interest will be integrated. The gene of interest is bracketed by terminal inverted repeats creating the transposon. These inverted repeats contain binding elements for transposase. pCMV-SB (SB), containing the transposase was provided by Dr. Perry Hacket (University of Minnesota). SB-TAg, containing the SV40 large T antigen under control of a CMV promoter, was provided by Dr. David Largaspada. For transfection, one well of a 6-well dish of confluent primary POSE cells 200,000 cells) was split 1:2 and allowed to grow to 60-80% confluence. This passage (referred to as passage 1) was used for subsequent transfection. Cells were transfected with a 3:1 ratio of SB-TAg:SB vectors using the cationic lipid LT1® (Promega Inc., Madison, Wis.) for 48 hours in complete media, according to manufacturers directions. After 48 hours of transfection cells were washed and allowed to recover for 48 hours before passage. Without being bound by theory, the T-Ag should confer a growth advantage to those cells expressing T-Ag, and also, at a minimum, immortalize the T-Ag expressing cells. This should allow the cells to grow and persist as the non-T-Ag expressing POSE cells simultaneously senesce. Therefore, the protocol was to repeatedly subculture the cells. In order to achieve better survival and growth, cells were cultured at high densities. A complete diagram of subculturing scheme is shown as FIG. 1.

### Validation of TAg Expression by Western Blot

Briefly, twenty μg cell lysate was subjected to electrophoresis on SDS-PAGE and Western blotting performed according to established protocols known in the art. T-Ag was detected using a mouse monoclonal antibody to SV40 large T-Ag (Pab101, 1:2000, Santa Cruz Biotechnology Inc., Santa Cruz, Calif.) followed by chemiluminescent detection. Blots were probed with anti-GAPDH (1:2500) as a loading control. A cell lysate from a T-antigen transformed murine ovarian carcinoma (MOVCAR) cell line was used as a positive control for detection of T-antigen.

### Verification of Cell Origin by Genomic PCR

Genomic DNA was isolated from POVCAR cells, POSE cells freshly isolated from pig ovaries, normal human ovarian surface epithelial (HOSE) cells, and normal mouse ovarian surface epithelial (MOSE) cells. These DNA samples were subjected to PCR as described (Corona et al. (2007) Span. J. Agric. Res., 5:312-317). The porcine specific primers are as follows:

(SEQ ID NO: 1)

Forward 5′ GCC TAA ATC TCC CCT CAA TGG TA 3′

(SEQ ID NO: 2)

Reverse 5′ ATG AAA GAG GCA AAT AGA TTT TCG 3′

Genomic GAPDH markers were used as loading control. The POVCAR cell DNA and POSE cell DNA were the only DNA samples capable of generating a PCR product with SEQ ID NOs: 1 and 2.

### Soft Agar Assay

2×10⁵ POVCAR cells were suspended in 3 ml of 0.35% sterile agarose (Ultrapure, Invitrogen, Carlsbad, Calif.) in complete media, and layered over 4 ml of 0.5% agarose as a support layer in 60 mm dishes. The dishes were incubated at 37° C. in 5% CO₂ in a humidified incubator for two weeks. Every five days, three to four drops of fresh media were added to the dishes. To visualize and quantitate colonies, dishes were fixed in 70% ethanol for 1 hour at room temperature then stained for 20 minutes with 0.1% methylene blue in 50% ethanol. Dishes were sufficiently destained with ddH₂0 and colonies counted on a Minicount counter (Imaging Products International Inc., Simi Valley, Calif.). Colonies of at least 50 cells were scored as colonies (0.3 mm). Microscopy was performed on a Nikon Stereo Dissecting Microscope (Nikon, Melville N.Y.) to obtain detailed images of individual colonies. Dishes were photographed using a digital camera. All images were processed using Adobe Photoshop software (Adobe Inc., Berkeley, Calif.).

### Tumorigenicity in Immunocompromised Mice

Female CB17 severe combined immunodeficient (SCID) mice, 8 weeks of age and weighing 20 g, were used to determine tumorigenicity of the transformed POVCAR cells. All mice were bred in the Laboratory Animal Facility at Fox Chase Cancer Center, maintained in specific pathogen-free conditions, and received commercial food and water ad libitum. Institutional guidelines, established by Fox Chase Institutional Animal Care and Use Committee, were used in handling of animals. POVCAR cells were grown to confluency, trypsinized, and washed in complete media. Cells were resuspended in sterile PBS and each mouse inoculated with 0.1 ml of cell suspension (5×10⁶ cells) subcutaneously (s.c.) bilaterally, on the flank. Mice were monitored for tumor growth every six days for three months. Mice which had tumors greater than 10% of body size were immediately euthanized. Flank tumors were fixed in buffered-formalin, paraffin embedded, and sectioned for hematoxylin/eosin staining and further immunohistochemical analysis for T-Ag and pan-cytokeratin (Sigma-Aldrich, St. Louis Mo.).

### Results

### Cell Isolation and Transfection

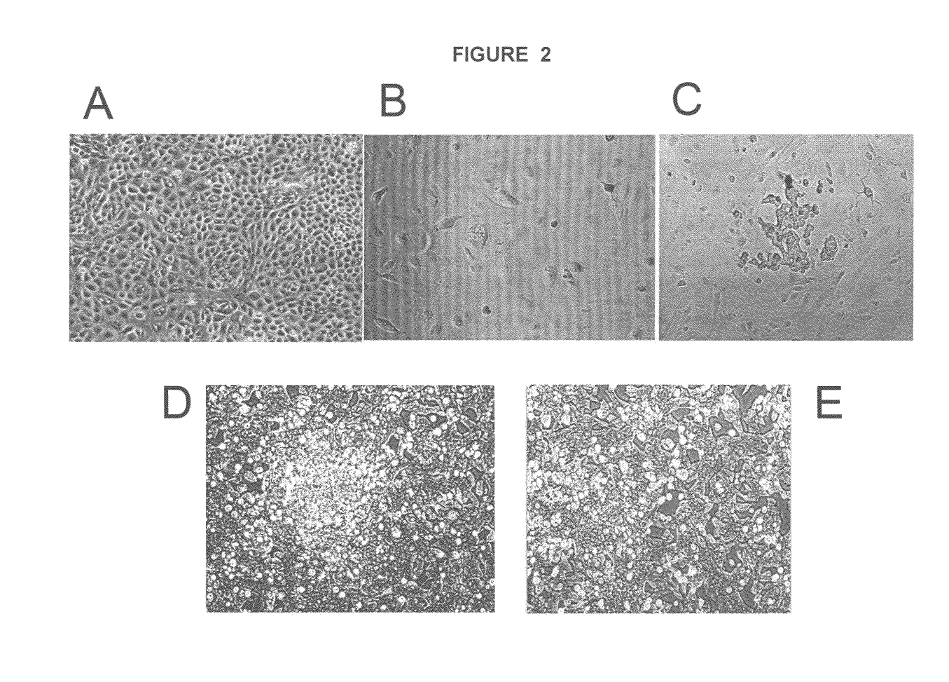

The scheme for subculturing the SB-Tag transfected POSE cells is depicted in FIG. 1. The second trypsinized population of POSE cells described in the materials and methods was used for transfection with the Sleeping Beauty-TAg system. More specifically, first, the primary POSE cells were initially cultured in one well of a 6-well dish (10 cm²/well), and the cells, with cobblestone epithelial morphology, are shown in FIG. 2A. When the cells reached near confluency, they were divided equally into two wells of a 6-well dish, which was considered passage 1. SB-TAg was transfected as described in the materials and methods of the example. The transfection reagent, LT1, resulted in significant cytotoxicity to the POSE cells and each well did not regain confluence. A majority of these cells were noticeably losing epithelial cell morphology and appearing to senesce. Therefore, both transfected wells of the 6-well dish were combined into one well of a 24-well dish (1.9 cm²/well) (Fisher Scientific). This was considered passage 2. Cells were allowed to grow, however, many appeared to senesce. Therefore, cells were passaged into a well of a new 24-well dish and FIG. 2B depicts the cells (passage 3). As shown in FIG. 2B, the majority of cells have lost the cobblestone morphology and appear to be senescing. This passaging was repeated an additional time (passage 4). At this point, there were noticeable groups of cells morphologically different from the majority of cells, presumably the POSE cells which had integrated T-Ag, and shown in FIG. 2C. Cells growing in the center of the micrograph were hypothesized to be outgrowths of the rare POSE cells that had been transfected with T-Ag. They have a more epithelial morphology and were able to grow without contact inhibition, providing morphologic support that they expressed T-Ag. These distinct cells showed a more cobblestone morphology, acquired a growth advantage compared to neighboring senescent cells, and formed small foci. To disperse the cells in the foci, cells were harvested with trypsin and plated in one well of a 6-well dish (passage 5). In this setting, the cells grew to confluence and many more foci formed as evidenced by FIG. 2D. FIG. 2D demonstrates the obvious loss of contact inhibition along with focus formation. After a further passage reached confluence, the majority of the cells were morphologically similar to the epithelioid cells seen in the original small foci and were clearly doubling at a reasonable rate as shown in FIG. 2E. Note the morphology similar to the cells growing in the center of FIG. 2C. This 10 cm² culture was harvested and placed in a T25 flask (25 cm², Fisher Scientific), and was considered passage 7.

### Testing for TAg Expression and the Species of Origin of the Cells

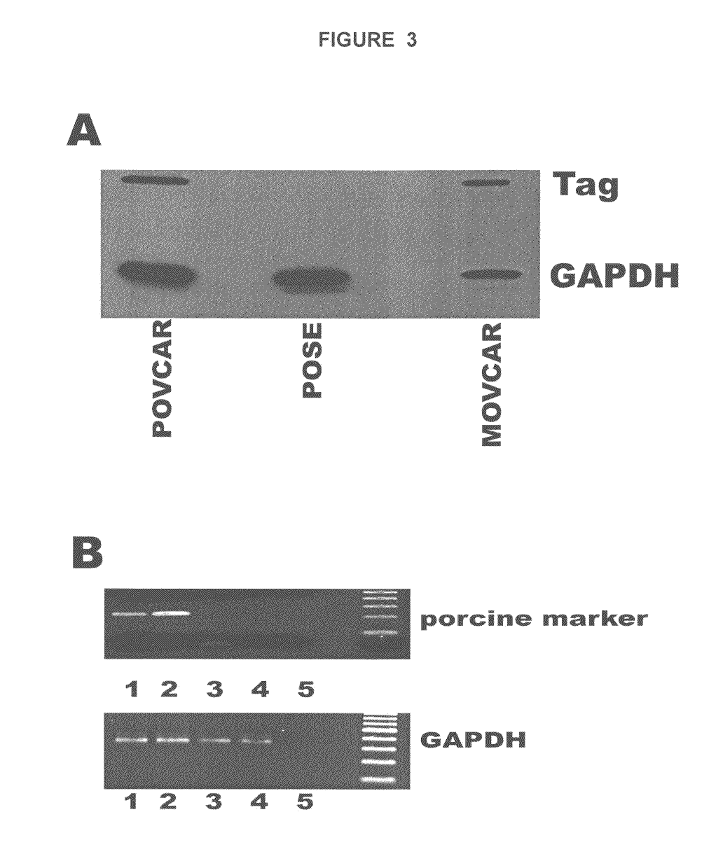

Western blotting was performed to confirm the presence of T-Ag in the cells harvested from passage 7, and the results are shown in FIG. 3A. The cells in lanes 1 and 3 were found to be T-Ag positive, and are referred to as POVCAR cells (Porcine OVarian CARcinoma cells). Passage 8 POVCAR cells clearly show the positive presence of T-Ag, while normal POSE cells show no T-Ag expression. To verify that the POVCAR cells were indeed of porcine origin, a genetic marker was used which is specific for the porcine species (Corona et al. (2007) Span. J. Agric. Res., 5:312-317), and the species of origin was verified by genomic PCR. Specifically, the results of the PCR experiments are shown in FIG. 3B, and lane 1 confirms the POVCAR cells are porcine in origin.

### Colony Formation in Soft Agar (Anchorage Independent Growth)

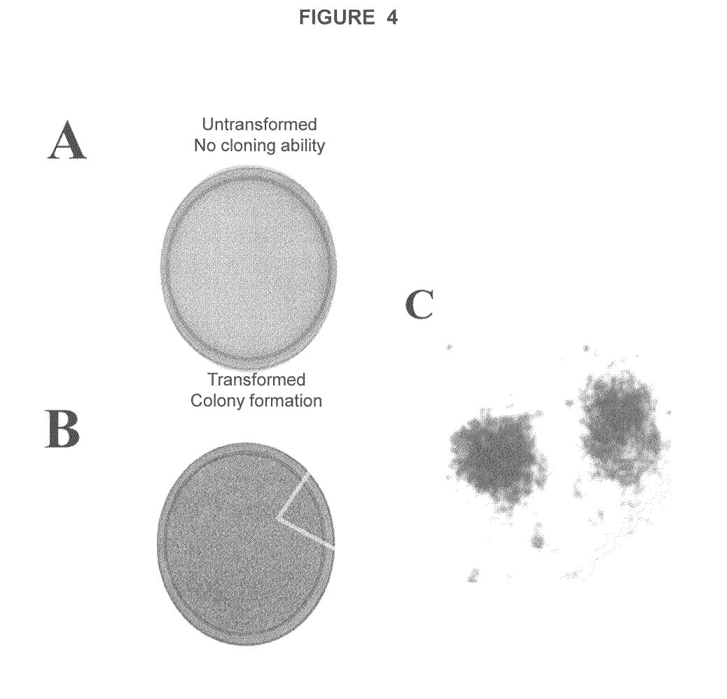

The ability to clone in soft agar is a hallmark of transformed cells and predictive of their tumorigenic potential. Thus, the ability of the POVCAR cells to grow in soft agar was assessed. As demonstrated in FIGS. 4B and 4C, the POVCAR cells display high efficiency (−0.2%) in forming colonies.

### Tumor Formation in SCID Mice

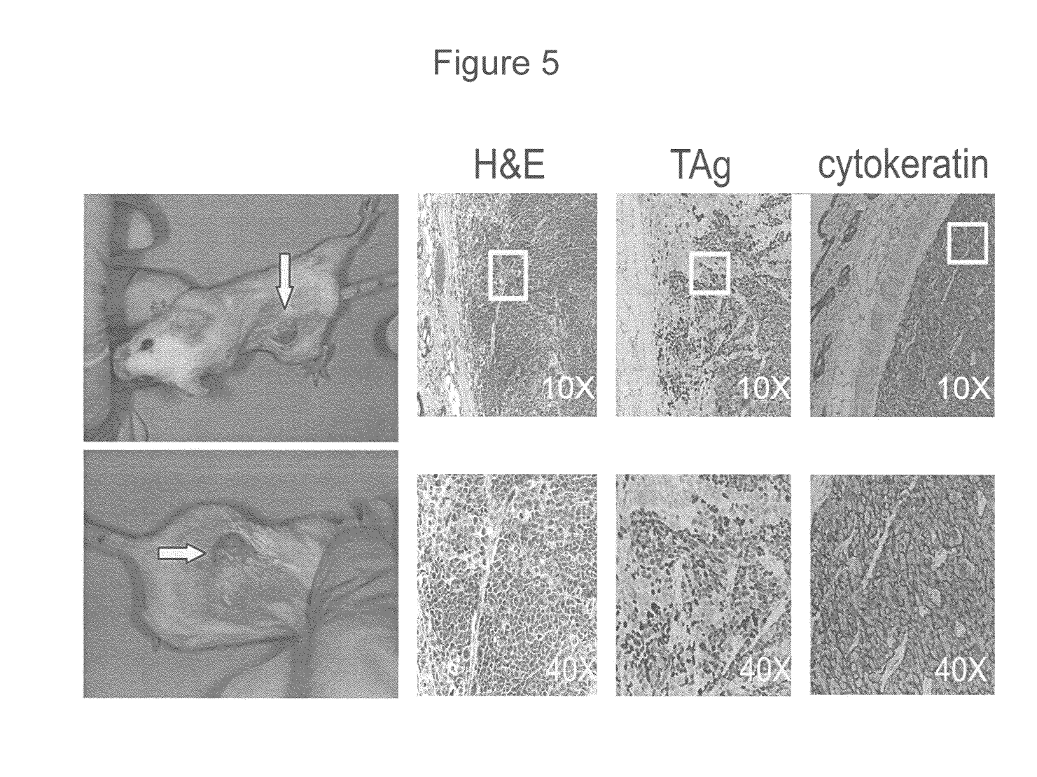

To assess the tumorigenic potential of the transformed POVCAR cells, a population of POVCAR cells grown to confluency were subcutaneously injected into the flank of SCID mice as described in the materials and methods. FIG. 5 (left panels) show tumor formation one month after POVCAR cell injection. Three months after innoculation, immunohistochemical analysis was performed, and the tumors were positive for cytokeratin (epithelial tissue marker) and T-Ag (transformed cell marker, FIG. 5 right panels).

### Permissivity of POVCAR Cells to Adenovirus Infection and Replication

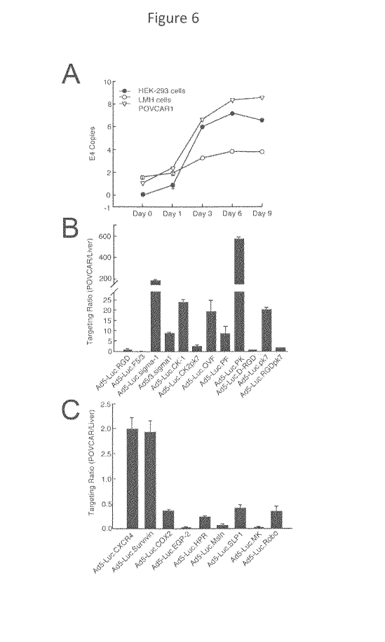

In order to confirm reports that adenovirus serotype 5 can infect and replicate in porcine cells, the POVCAR cells were first infected with wild-type adenovirus and the resulting production of progeny virions generated over a period of nine days analyzed. This was performed in comparison to human embryonic kidney 293 cells (HEK-293), which are known to be permissive to replication and are commonly used to propagate adenovirus. In this experiment, a chicken cell line, LMH, was also included for comparison, as the literature has noted adenovirus replication permissiveness in chicken cells. Over a period of nine days, adenoviral replication was highest in the POVCAR cells (FIG. 6A) and lowest in the chicken cell line LMH.

The use of conditionally replicating adenovirus (CRAds) as an oncolytic agent to treat ovarian cancer is limited by not only the lack of the native Ad receptor on the tumor or target cells but also the native tropism of Ad to the liver which confounds therapy due to associated hepatotoxicity. To address these issues, we will employ a panel of infectivity enhanced adenovirus agents in which the capsid proteins are modified to increase infectivity in the tumor or target cells. In FIG. 6B, the ability of these viruses to preferentially infect POVCAR cells and not infect porcine liver was investigated. Each virus was evaluated and compared to unmodified Ad and expressed as a ratio of targeting the POVCAR cells divided by the liver expression. Significant targeting gains were accrued with many of the capsid modifications but strikingly so with the adenovirus containing the knob domain from porcine adenovirus (Ad5-Luc.PK).

To further limit Ad associated hepatotoxicity, cell type specific promoters have been utilized to decrease replication and transgene expression in the liver. To test the ability of candidate promoters to prevent expression in the liver yet still maintain expression and replication in the POVCAR cells, POVCAR cells were infected with native adenoviral capsid vectors containing each promoter controlling expression of the reporter gene luciferase. Utilizing a targeting ratio, as above, the relative abilities of the promoters to prevent expression in the liver and maintain expression in the POVCAR cells were determined (FIG. 6C). These data implicate that two strong candidates, the CXCR4 and survivin promoters are useful for this purpose.

While certain of the preferred embodiments of the present invention have been described and specifically exemplified above, it is not intended that the invention be limited to such embodiments. Various modifications may be made thereto without departing from the scope of the present invention, as set forth in the following claims.

What is claimed is:

1. A method for producing an animal model of cancer, said method comprising:
a) isolating a population of cells from a donor animal,
b) transforming said population of cells with a nucleic acid molecule,
c) culturing said transformed population of cells, and
d) injecting the transformed cells of step c) into a recipient animal, thereby producing said animal model of cancer.

2. The method of claim 1, wherein said cancer is ovarian cancer.

3. The method of claim 2, wherein said population of cells of step a) comprise ovarian epithelial cells.

4. The method of claim 1, wherein said nucleic acid molecule encodes SV40 T-Ag.

5. The method of claim 1, wherein the recipient animal is the same species as the donor animal.

6. The method of claim 1, wherein the recipient animal is a different species than the donor animal.

7. The method of claim 1, wherein the donor animal is a pig and the population of cells of step a) are porcine ovarian surface epithelial cells.

8. The method of claim 1, wherein the recipient animal is a pig.

9. The method of claim 8, wherein said pig is an inbred miniature swine.

10. The method of claim 1, wherein step b) is performed with the Sleeping Beauty T-Ag transposon system.

11. The animal produced according to the method of claim 1.

12. An animal comprising an allograft or xenograft of an ovarian carcinoma, wherein said ovarian carcinoma comprises ovarian epithelial cells transformed with SV40 T antigen.

13. The animal of claim 12 which is porcine.

14. The method of claim 5, wherein said recipient animal and said donor are genetically identical clones.

15. The method of claim 14, wherein said clones are obtained from somatic cell nuclear transfer.

16. A method for identifying at least one active agent for cancer therapy comprising:
a) providing the animal of claim 11,
b) administering said at least one agent to said animal, and
c) determining the effect of the agent(s) on the tumor of the animal,
wherein a decrease in the size of the size of the tumor compared to a control animal not administered the agent(s) indicates the administered agent is an active agent for cancer therapy.

17. The method of claim 16, wherein the agent comprises an oncolytic conditionally replicative adenovirus.

18. A method for identifying at least one chemotherapeutic agent comprising:
a) providing the animal of claim 11, and
b) administering an agent(S) to said animal,
wherein an increase in the survival of the animal of step b) compared to a control animal not administered the agent indicates the administered agent is a chemotherapeutic agent.

19. The method of claim 18, wherein the agent comprises an oncolytic conditionally replicative adenovirus.

20. The method of claim 18, wherein said at least one chemotherapeutic agent is selected from the group consisting of mechlorethamine, cyclophosphamide, ifosfamide, melphalan, chlorambucil, carmustine (BCNU), lomustine (CCNU), semustine (methyl-CCNU), Ethylenimine/Methylmelamine, thriethylenemelamine (TEM), triethylene thiophosphoramide (thiotepa), hexamethylmelamine (HMM), altretamine busulfan, dacarbazine (DTIC), methotrexate, trimetrexate, 5-fluorouracil, fluorodeoxyuridine, gemcitabine, cytosine arabinoside, 5-azacytidine, 2,T-difluorodeoxycytidine, —6-mercaptopurine, 6-thioguanine, azathioprine, T-deoxycoformycin (pentostatin), erythrohydroxynonyladenine (EHNA), fludarabine phosphate, 2-Chlorodeoxyadenosine (cladribine, 2-CdA)), camptothecin, topotecan, irinotecan, paclitaxel, vinblastine (VLB), vincristine, and vinorelbine, Taxotere®,docetaxel, estramustine, estramustine phosphate, etoposide, teniposide, doxorubicin (adriamycin), mitoxantrone, idarubicin, bleomycins; plicamycin (mithramycin), mitomycinC, dactinomycin, L-asparaginase, interferon-alpha, IL-2, G-CSF, GM-CSF, retinoic acid derivatives, metronidazole, misonidazole, desmethylmisonidazole, pimonidazole, etanidazole, nimorazole, RSU 1069, E09, RB 6145, SR4233, nicotinamide, 5-bromodeozyuridine, 5-iododeoxyuridine, bromodeoxycytidine, cisplatin, carboplatin, mitoxantrone, hydroxyurea, N-methylhydrazine (MIH), procarbazine, aminoglutethimide, interferon β, interferon γ, interleukin-2, prednisone, dexamethasone, aminoglutethimide, hydroxyprogesterone caproate, medroxyprogesterone acetate, megestrol acetate, diethylstilbestrol, ethynyl estradiol, tamoxifen, testosterone propionate, fluoxymesterone, flutamide, leuprolide, hematoporphyrin derivatives, Photofrin®, benzoporphyrin derivatives, Npe6, tin etioporphyrin (SnET2), pheoboride-α, bacteriochlorophyll-a, naphthalocyanines, phthalocyanines, zinc phthalocyanines, bortezomib (Velcade®), epothilone, serratamolide, imatinib mesylate, dasatinib, nilotinib, MK-0457, and Omacetaxine, cetuximab, remicade, and herceptin.


SEQUENCE LISTING (2 sequences)

SEQ ID NO: 1 - DNA, 23 bp, Artificial Sequence
  Primer
gcctaaatctcccctcaatggta

SEQ ID NO: 2 - DNA, 24 bp, Artificial Sequence
  Primer
atgaaagaggcaaatagattttcg